(19) United States
(12) Patent Application Publication
(10) Pub. No.: US 2003/0091601 A1
(43) Pub. Date: May 15, 2003

(54) Use of topical arginine to enhance wound healing

(75) Inventor: Adrian Barbul, Baltimore, MD (US)

Correspondence Address: Dr. Adrian Barbul, 302 Club Road, Baltimore, MD, 21210, US

(73) Assignee: ABAT Inc., Baltimor, MD (US)

(21) Appl. No.: 10/054,229

(22) Filed: Nov. 13, 2001

(51) Int. Cl. A61K 31/198

(52) U.S. Cl. 424/400; 514/565

(57) ABSTRACT

The present invention relates to the treatment of wounds by the topical administration of arginine or ornithine to wound sites. There is described herein a wound healing enhancing medicinal composition formulated from the amino acid arginine or a by-product thereof. Topical application of the medicinal composition to the wound site enhances wound healing by increasing collagen biosynthesis. A medical composition of the present invention may be administered alone, on dressings, or in combination with common vehicles for topical application including, for example, oil-based emulsions, gels, creams, ointments, and the like.

### BACKGROUND OF THE INVENTION

1. Field of the Invention

This invention relates generally to a method of enhancing wound healing. More specifically, the present invention relates to the isolation and use of the amino acid arginine or any derivative amino acid products of arginine catabolism via the urea cycle, in a topical application in the treatment of wound or lesion healing.

### BACKGROUND OF THE PRIOR ART

Wound healing is a complex process involving factors such as cells, extracellular matrix components and the cellular microenvironment. Essentially, all wound healing involves the repair or replacement of damaged tissues, including but not limited to skin, muscle, neurologic tissue, bone, soft tissue, internal organs or vascular tissue. The precise nature of such repair or replacement depends upon the tissues involved, although all such processes involve certain basic principles. An important aspect of wound healing is the rate at which a wound gains tensile strength against breakage.

Skin exhibits tension. Skin tension is one of the determining factors in the response to a wound and varies with age and site. Skin has multiple layers, including a fibrous network composed of collagen and elastin. If only the epidermis layer of skin is damaged, as in most minor injuries, keratinocytes migrate from the edge of the wound and eventually cover it, reforming the epidermis (Knighton, D. R. and Fiegel, V. D., 1991, Invest. Radiol: 26:604-611).

If all skin layers are injured, new connective tissue must first fill in the wound space. Such tissue, identified as granulation tissue, is formed by deposition of extracellular matrix components such as collagen, by fibroblasts which migrate into the wound space. The synthesis and deposition of collagen is an important event in wound healing and the rate of collagen synthesis varies in different organs (Haukipuro, K. et al., 1991, Ann. Surg. 213: 75-80).

Various treatments have been used to accelerate the rate at which wounds heal. A present method relates to providing a nutritional formulation including an arginine source in an amount of at least 2% of the total calories of the composition. The method does not teach or suggest using arginine in a pharmaceutical carrier to topically deliver arginine to the wound. Another method for aiding in wound healing includes the administration of a pharmaceutical composition comprising a peptide having more than three consecutive basic amino acids. The method does not teach or suggest administering a pharmaceutical composition comprising arginine as the sole active ingredient in enhancing wound healing.

Alternate present treatments in enhancing wound healing include the application of therapeutic microsphere compositions containing wound healing agents for topical application to wounds and/or lesions for accelerating muscle regeneration. Such treatment specifically teaches the addition of a growth factor in combination with L-arginine as an immune enhancer and does not suggest a composition solely comprising the amino acid arginine or an arginine by-product as the effective wound healing agent. Alternatively, a further method for treating wounds provides a topical gel formulation wherein the gel includes a growth factor and arginine. This method, however, is particularly directed toward the anterior chamber of the eye and does not suggest or teach the use of the amino acid arginine or its by-products in enhancing wound healing.

As noted earlier, collagen biosynthesis at a wound site is a necessary step in promoting wound healing. Studies have shown that in the wound environment, arginine concentrations are very low and in the latter phases of wound healing, when fibroplasia is at a maximal level, arginine concentrations are non-detectable or extremely low (Albina, J. E. et al. Arginine metabolism in wounds. Am J Phys 1988; 254: E459). Such studies suggest that arginine does not play a vital role in wound healing as a substrate for collagen biosynthesis. Contrary to such studies, my recent research directed to the biological effects of topically applied arginine or its by-product to wounds has demonstrated unique beneficial effects toward enhancing wound healing. Consequently, there exists a need to develop methods for using arginine or its by-products in topical application to wound sites.

### SUMMARY OF THE INVENTION

The present invention provides a unique use of arginine toward enhancing wound healing. Specifically, the present invention teaches the topical application of the amino acid arginine or its by-products, such as omithine, to wound sites to promote enhanced increased wound collagen deposition and enhanced strength against wound breaking.

Arginine has been classified as being one of two semi-essential amino acids in mammalian metabolism. This amino acid has been identified as a wound healing factor in a nutritionally fortified pharmaceutical composition. The amino acid of this invention, it is believed, is involved in the healing process to synthesize collagen and polyamines.

Supplemental arginine is a precursor for polyamines, which are critical biomolecules in situations of enhanced cellular proliferation. Ornithine represents the first major metabolic precursor for polyamine biosynthesis and it has been hypothesized that the beneficial effects of supplemental arginine on wound healing may occur

An object of the invention is to provide a method for topical application of the amino acid arginine to patients with an acute or chronic wound comprising the step of administering a therapeutically effective amount of a composition. The composition includes a unique amino acid profile.

Another object of the invention is that it minimizes complications in post-surgical patients.

Another object of the invention is that it reduces the financial burden to institutions and health care systems as a result of less wound care time, decreased complications and possibly less rehospitalization.

Another object of the invention is that it promotes increased wound collagen synthesis and deposition. This in turn contributes to better wound repair. In addition, it prevents wound breaking.

Still another object of the invention is to decrease the morbidity rate for patients having chronic or acute wound sites.

These and other features, aspects and advantages of the present invention will become better understood with reference to the following description and appended claims.

### DETAILED DESCRIPTION

The invention summarized above and defined by the enumerated claims may be better understood by referring to the following detailed description. This detailed description of a particular preferred embodiment, set out below to enable one to practice the invention, is not intended to limit the enumerated claims, but to serve as a particular example thereof. Those skilled in the art should appreciate that they can readily use the concepts and specific embodiment disclosed as a basis for modifying or designing other methods and systems for carrying out the same purposes of the present invention. Those skilled in the art should also realize that such equivalent methods and systems do not depart from the spirit and scope of the invention in its broadest form.

The present invention provides a means for enhancing wound healing in a subject by administering to the subject a pharmaceutical composition comprising a therapeutically effective amount of the amino acid arginine or a derivative of arginine, such as ornithine or the like. The present invention provides pharmaceutical compositions and methods for topical treatment of wounds with demonstrable efficacy in the promotion of collagen synthesis and wound healing. The pharmaceutical composition taught herein include a unique profile containing either arginine or a by-product of arginine, such as ornithine or the like.

With respect to the treatment aspect, the present invention can be utilized to treat patients with wounds, in particular patients with acute and/or chronic wounds. Among others, such patients include, for example, Type I diabetics with necrotizing fascitis, nursing home patients with decubitus ulcers, hospitalized and homecare post-surgical patients with acute or chronic wounds, long-term tube fed patients suffering from a wound, and subacute care patients suffering from a wound. Specifically, the present invention provides a means for providing an effective topical application for wound healing patients in an acute or long term care setting.

Pursuant to the present invention, a formulation is provided that is designed to optimize collagen synthesis and wound healing in patients, in particular patients with with acute and chronic wounds. The formulation of the present invention provides a novel pharmaceutical formulation for topical application toward enhancing the healing of acute and chronic wounds. The formulation can be provided alone, on dressings, or in combination with ointments, emollients, and growth factors.

An important phase of wound healing is the formulation of fibroblast and collagen. The inventor has surprisingly discovered that the topical application of the amino acid arginine results in a significant improvement in the parameters used to gauge wound healing. The parameters include such factors as wound breaking strength and collagen synthesis.

Although arginine is not considered to be an essential amino acid in wound healing, it is proposed that arginine is a substrate for collagen biosynthesis in the wound healing process. More importantly, it is proposed that arginine is a precursor for polyamines, which are critical biomolecules in situations of enhanced cellular proliferation.

By way of example, and not limitation, experimental examples detailing the use of the present invention will be provided below.

### EXPERIMENTAL EXAMPLE 1

Twenty-eight (28) male adult rats were obtained from a commercial breeder. Rats were housed individually in stainless steel cages at a constant temperature (25°±1° C.) and relative humidity (40-50%). The cages were kept in a room with a twelve-hour light-twelve-hour dark cycle. All animals were allowed at least one week of acclimatization to the laboratory conditions prior to use in the experiments. During this time, they were fed a standard laboratory chow and drank tap water ad libitum.

After the period of acclimatization, the rats were randomly placed into four groups, seven rats per group. Under pentobarbital anesthesia, their backs were shaved and scrubbed with an organic iodine solution and a dorsal skin incision was made. Subcutaneously at the skin incision, a mini-osmotic pump (model 2002, 0.5 μl/hr, Alzet Co.) attached to a polyvinyl alcohol sponge was inserted. Pumps were filled either with PBS or various solutions of arginine in PBS as indicated on Table 1. The rats tolerated the procedure well and recovered uneventfully.

TABLE 1

| Group | Treatment |
|---|---|
| 1 | PBS |
| 2 | Arginine 1 M |
| 3 | Arginine 0.1 M |
| 4 | Arginine 0.01 M |

The 1M solution delivers 2 mg/animal/day into the sponge. On the fourteenth day post-wounding, rats were sacrificed with an overdose of pentobarbital. The pelt containing the healing scar was excised. The PVA sponges were removed, carefully cleared of surrounding tissue and frozen at −20° C. for subsequent determination of hydroxyproline content as an index of wound reparative collagen accumulation using the method of Woessner. See J. Woessner, Determination of Hydroxproline in Tissue and Protein Samples Containing Small Proportions of this Amino Acid, Biochem. Biophys., Vol.93, p. 440 (1961).

### Results

Table 2 below sets forth results for the wound healing parameters that were evaluated in the experiment. All data are reported as mean±SEM. Statistical analysis was carried out using the StaView Program on a Macintosh Computer and applying analysis of variance (ANOVA) with Scheffe's F-test being applied for post hoc analysis.

TABLE 2

| | | OHP |
|---|---|---|
| Group | Treatment | (μg/100 mg sponge) |
| 1 | PBS | 569 ± 27 |
| 2 | Arginine 1 M | 928 ± 55* |
| 3 | Arginine 0.1 M | 698 ± 74 |
| 4 | Arginine 0.01 M | 633 ± 54 |

Legend
*p < 000.1 by ANOVA
OHP = collagen synthesis

### Conclusions

The data illustrate that the highest dose of locally infuse arginine significantly increases wound collagen deposition. Most notably, the wound healing study revealed that topical application of arginine at 1M to a wound site enhances collagen biosynthesis accumulation by over 60% when compared to the PBS (control) group. In addition, topical application of lower dosages of arginine, at 0.1M and 0.01M resulted in at least a 10% increase in collagen biosynthesis accumulation as compared to the PBS (control) group. The interpretation of these data would suggest that topical application of arginine to a wound site has a superior effect in enhancing wound healing. Although the exact mechanism of action remains to be elucidated, topical application of 1M of arginine to a wound site resulted in over a 60% increase in wound collagen deposition.

### EXPERIMENTAL EXAMPLE 2

The inventor conducted an additional experiment to demonstrate the efficacy of the topical application of arginine to a wound site at a dose of 1M. Twenty (20) male adult rats were obtained from a commercial breeder. As in Experimental Example 1, rats were housed individually in stainless steel cages at a constant temperature (25°±1° C.) and relative humidity (40-50%). The cages were kept in a room with a twelve hour light-twelve hour dark cycle. All animals were allowed at least one week of acclimatization to the laboratory conditions prior to use in the experiments. During this time, they were fed a standard laboratory chow and drank tap water ad libitum.

After the period of acclimatization, the rats were randomly placed into two groups of ten rats. Under pentobarbital anesthesia, the rats' backs were shaved and scrubbed with an organic iodine solution and a dorsal skin incision was made. As in Experimental Example 1, a mini-osmotic pump (model 2002, 0.5 μl/hr, Alzet Co.) attached to a polyvinyl alcohol sponge was inserted subcutaneously at the incision site. Pumps were filled either with PBS or a solution of 1M arginine in PBS as indicated on Table 3. The rats tolerated the procedure well and recovered uneventfully.

TABLE 3

| Group | Treatment |
|---|---|
| 1 | PBS |
| 2 | Arginine 1 M |

The 1M solution delivers 2 mg/animal/day into the sponge. On the fourteenth day post-wounding, rats were sacrificed with an overdose of pentobarbital. The pelt containing the healing scar was excised. The PVA sponges were removed, carefully cleared of surrounding tissue and frozen at −20° C. for subsequent determination of hydroxyproline content as an index of wound reparative collagen accumulation using the method of Woessner. See J. Woessner, Determination of Hydroxproline in Tissue and Protein Samples Containing Small Proportions of this Amino Acid, Biochem. Biophys., Vol.93, p. 440 (1961).

### Results

Table 4 below sets forth results for the wound healing parameters that were evaluated in the experiment. All data are reported as mean±SEM. Statistical analysis was carried out using the StaView Program on a Macintosh Computer and applying analysis of variance (ANOVA) with Scheffe's F-test being applied for post hoc analysis.

TABLE 4

| | | OHP |
|---|---|---|
| Group | Treatment | (μg/100 mg sponge) |
| 1 | PBS | 574 ± 43 |
| 2 | Arginine 1 M | 797 ± 48* |

Legend
*p < 0.05 by ANOVA
OHP = collagen synthesis

### Conclusions

The data illustrate that the highest dose of locally infuse arginine significantly increases wound collagen deposition. Most notably, the wound healing study affirmed the results garnered from Experimental Example 1 and suggests that topical application of arginine at 1M to a wound site dramatically enhances collagen biosynthesis accumulation by over 30% when compared to the PBS (control) group. The interpretation of these data would suggest that topical application of arginine to a wound site has a superior effect in enhancing wound healing.

### EXPERIMENTAL EXAMPLE 3

A further experiment was conducted by the inventor to demonstrate the efficacy of omithine, an amino acid which is the product of arginine catabolism via the urea cycle. An objective of the present experiment was to establish the effect of omithine on collagen deposition against a (control) group where wound sites were applied with only a PBS solution. The experiment was performed to determine whether topical application of by-products of the amino acid arginine were as effective as arginine itself in enhancing wound healing.

Twenty-one (21) male adult rats were obtained from a commercial breeder. As in Example Experiments 1 and 2, rats were housed individually in stainless steel cages at a constant temperature (25°±1° C.) and relative humidity (40-50%). The cages were kept in a room with a twelve hour light-twelve hour dark cycle. All animals were allowed at least one week of acclimatization to the laboratory conditions prior to use in the experiments. During this time, they were fed a standard laboratory chow and drank tap water ad libitum.

After the period of acclimatization, the rats were randomly placed into two groups, eight (8) rats were placed into Group 1 and treated with PBS only and thirteen (13) rats were placed into Group 2 and treated with 1M omithine in PBS. Under pentobarbital anesthesia, the rats' backs were shave and scrubbed with an organic iodine solution and a dorsal skin incision was made. Subcutaneously at the skin incision, a mini-osmotic pump (model 2002, 0.5 μl/hr, Alzet Co.) attached to a polyvinyl alcohol sponge was inserted. The mini-osmotic pumps were filled either with PBS or a solution of 1M omithine in PBS as indicated on Table 5 below. The rats tolerated the procedure well and recovered uneventfully.

TABLE 5

| Group | Treatment |
|---|---|
| 1 | PBS |
| 2 | Ornithine 1 M |

The 1M omithine solution was delivered to the animals at the rate of 0.5 μg/hour/14 days. On the fourteenth day post-wounding, rats were sacrificed with an overdose of pentobarbital. As with Example Experiments 1 and 2, the pelt containing the healing scar was excised. The PVA sponges were removed, carefully cleared of surrounding tissue and frozen at −20° C. for subsequent determination of hydroxyproline content as an index of wound reparative collagen accumulation using the method of Woessner. See J. Woessner, Determination of Hydroxproline in Tissue and Protein Samples Containing Small Proportions of this Amino Acid, Biochem. Biophys., Vol.93, p. 440 (1961).

### Results

Table 6 below sets forth results for the wound healing parameters that were evaluated in the experiment. All data are reported as mean±SEM. Statistical analysis was carried out using the StaView Program on a Macintosh Computer and applying analysis of variance (ANOVA) with Scheffe's F-test being applied for post hoc analysis.

TABLE 6

| | | OHP |
|---|---|---|
| Group | Treatment | (μg/100 mg sponge) |
| 1 | PBS | 588 ± 45 |
| 2 | Ornithine 1 M | 854 ± 71* |

Legend
*p < 0.05 by ANOVA
OHP = collagen synthesis

### Conclusions

The data illustrate that the dose of locally infuse ornithine significantly increases wound collagen deposition. Most notably, the wound healing study revealed that topical application of omithine, an arginine by-product, at 1M to a wound site enhances collagen biosynthesis accumulation by over 45% when compared to the PBS (control) group. The interpretation of the data would suggest that topical application of arginine by-products, such as ornithine, to a wound site has a superior effect in enhancing wound healing. Although the exact mechanism of action remains to be elucidated, topical application of omithine, a by-product of the amino acid arginine catabolism via the urea cycle, to a wound site resulted in over a 45% increase in wound collagen deposition.

It should be understood that although the invention has been described with reference to the examples provided above, various modifications can be made without departing from the spirit of the invention. It is therefore intended that the protection granted hereon be limited only be definition contained in the appended claims and equivalents thereof.

I claim:

1. A medicinal composition for enhancing the wound process in humans consisting essentially of a therapeutic wound healing enhancing effective amount of the amino acid arginine in a pharmaceutically acceptable vehicle for topical application.

2. The composition of claim 1, wherein the therapeutic wound healing enhancing effective amount of the amino acid arginine comprises at least 1M of arginine.

3. The composition of claim 1, wherein the pharmaceutically acceptable vehicle for the topical application is selected from the group consisting of:
(a) an oil-based emulsion;
(b) a gel;
(c) a cream; and
(d) an ointment.

4. The composition of claim 3, wherein the pharmaceutically acceptable vehicle is an oil-based emulsion.

5. The composition of claim 3, wherein the pharmaceutically acceptable vehicle is a gel.

6. The composition of claim 3, wherein the pharmaceutically acceptable vehicle is a cream.

7. The composition of claim 3, wherein the pharmaceutically acceptable vehicle is an ointment.

8. A medicinal composition for enhancing the wound process in humans consisting essentially of a therapeutic wound healing enhancing effective amount of the amino acid arginine catabolism by-product in a pharmaceutically acceptable vehicle for topical application.

9. The composition of claim 4, wherein the amino acid arginine catabolism by-product is omithine.

10. The composition of claim 4, wherein the therapeutic wound healing enhancing amount of the amino acid arginine catabolism by-product comprises at least 1M of the by-product.

11. The composition of claim 4 wherein the pharmaceutically acceptable vehicle for topical application is selected from the group consisting of:
(a) an oil-based emulsion;
(b) a gel;
(c) a cream; and
(d) an ointment.

12. The composition of claim 11, wherein the pharmaceutically acceptable vehicle is an oil-based emulsion.

13. The composition of claim 11, wherein the pharmaceutically acceptable vehicle is a gel.

14. The composition of claim 11, wherein the pharmaceutically acceptable vehicle is a cream.

15. The composition of claim 11, wherein the pharmaceutically acceptable vehicle is an ointment.

16. A method for enhancing the healing of wounds comprising the topical application to a wound site a therapeutically wound healing enhancing effective amount of the composition of claim 1.

17. The method for enhancing the healing of wounds of claim 16, wherein the composition of claim 1 comprises at least 1M of the amino acid arginine.

18. The method for enhancing the healing of wounds of claim 16, wherein the pharmaceutically acceptable vehicle for the topical application of claim 1 is selected from the group consisting of:
(a) an oil-based emulsion;
(b) a gel;
(c) a cream; and
(d) an ointment.

19. A method for enhancing the healing of wounds comprising the topical application to a wound site a therapeutically wound healing enhancing effective amount of the composition of claim 4.

20. The method for enhancing the healing of wounds of claim 18, wherein the therapeutic wound healing enhancing amount of the composition of claim 4 comprises at least 1M of the amino acid arginine catabolism by-product.
